(19) United States
(12) Patent Application Publication
(10) Pub. No.: US 2017/0105878 A1
(43) Pub. Date: Apr. 20, 2017

(54) Bandage Assembly

(71) Applicant: Carlton Parrish, Walters, OK (US)

(72) Inventor: Carlton Parrish, Walters, OK (US)

(21) Appl. No.: 15/299,149

(22) Filed: Oct. 20, 2016

Related U.S. Application Data
(60) Provisional application No. 62/243,810, filed on Oct. 20, 2015.

(51) Int. Cl. A61F 13/00; A61L 15/44; A61F 13/02

(52) U.S. Cl. CPC A61F 13/00063; A61F 13/023; A61F 13/025; A61L 15/44; A61F 2013/00157; A61F 2013/0028; A61F 2013/00182; A61L 2300/106; A61L 2300/104

(57) ABSTRACT

A bandage assembly for treating a patient's wound can consist of outer, inner, and adhesive layers. The outer layer may be constructed of a first material and have a window portion that is a semitransparent second material. The inner layer can be suspended across the window portion and be coated with at least one medication material. The adhesive layer may be disposed between and contact the outer layer as well as skin of a patient to apply continuous pressure on the inner layer.

### RELATED APPLICATION

The present application makes a claim of domestic priority to U.S. Provisional Patent Application No. 62/243,810 filed Oct. 20, 2015, the contents of which are hereby incorporated by reference.

### SUMMARY

A bandage assembly can treat a variety of different wounds, such as burns and cuts. A bandage assembly has outer, inner, and adhesive layers. The bandage assembly, in accordance with assorted embodiments, has an outer layer with a window portion. The outer layer consists of a first material and the window portion consists of a semitransparent second material. An inner layer is suspended across the window portion and is coated with at least one medication material. An adhesive layer is disposed between and contacts the outer layer and skin of a patient to apply continuous pressure on the inner layer.

### BRIEF DESCRIPTION OF THE DRAWINGS

FIG. 1 represents a portion of an example burn bandage system configured in accordance with assorted embodiments.

FIGS. 2A and 2B display different views of a portion of an example burn bandage assembly capable of being used in the burn bandage system of FIG. 1.

FIG. 3 illustrates a line representation of an example burn bandage assembly constructed and utilized as part of the burn bandage system of FIG. 1.

FIG. 4 depicts a line representation of an example burn bandage assembly capable of being used in the burn bandage system of FIG. 1.

FIG. 5 shows a line representation of a portion of an example burn bandage system configured in accordance with assorted embodiments.

FIG. 6 is a flowchart of an example burn treatment routine that is executed in accordance with various embodiments with the burn bandage system of FIGS. 1-5.

### DETAILED DESCRIPTION

There is a continued need for improved treatment systems for injuries where time is of the essence. When a patient is burned, keeping the injured area moist and free of contaminants is paramount to successful treatment. However, many burn injuries occur in locations where conventional burn treatments are insufficient to properly protect and moisturize a burn injury. For example, skin injuries and burns that occur in war zones or in war-ravaged locations can result in long and rough transport to a medical facility, which conventional bandages are not designed to endure.

Thus, assorted embodiments are directed to a burn bandage system with protective outer layer that seals an injury with an adhesive and applies pressure on an inner layer that is coated with an ointment or gel. By configuring the outer layer to seal an injury and continually apply pressure on the inner layer mitigates the risk of injury contamination and trauma while the inner layer continually provides moisture to the injured area. The ability to pre-package the outer, inner, and adhesive layers together prior to an injury occurring allows for efficient preparation and placement of the burn bandage assembly, which can be particularly important in hostile environments where weather or combat conditions can exacerbate a skin injury very quickly. In addition, the ease of preparation and installation of the pre-packaged burn bandage assembly allows non-medically trained personnel to utilize the treatment system.

With various embodiments, a burn bandage system provides protection of a burned area and sustains the tissue until the injured patient can be treated further at a treatment facility. The burn bandage system provides better protection from infection and outside contaminants, quicker and simpler application, and improved tissue hydration to minimize tissue damage upon removal at a treatment facility. The burn bandage system can consist of at least three elements: a sterile waterproof outer layer, a sterile non-stick emulsion pad, and a treatment material that may be an ointment or gel. The burn bandage can be held in place by any number of retention features, such as adhesive and/or compression fabric.

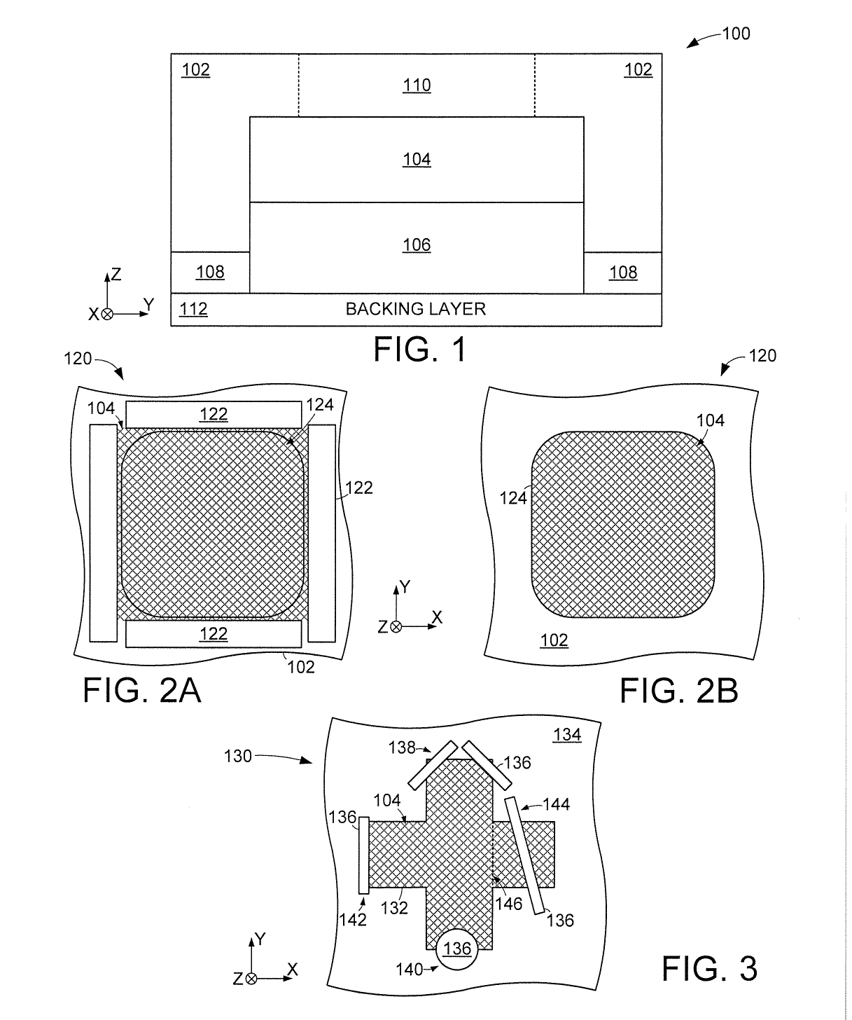

Turning to the drawings, FIG. 1 is a cross-sectional line representation of an example burn bandage system 100 configured in accordance with some embodiments. As shown, an outer layer 102 continually extends across and seals an emulsion pad 104 that is coated with, or suspends, a treatment material 106, such as microbial silver chloride ointment. The outer layer 102 has one or more adhesive layers 108 that are configured as one or more separate components that temporarily affix the outer layer to a patient's skin.

In some embodiments, the outer layer 102 window 110 that is made of a tinted, semitransparent, clear, or translucent material that allows the burn bandage system 100 to be aligned with an underlying burned region. It is contemplated that a majority, or the entirety of the outer layer 102 is made from a tinted, semi-transparent, or translucent material that is sterile and waterproof. The outer layer 102 may be a combination of multiple different materials with different densities and/or elasticity that can apply a predetermined amount of pressure onto the emulsion pad 104 and treatment material 106. For instance, a first outer layer 102 material can have less elasticity and be placed at the periphery of the window 110 portion of the outer layer 102 that has greater elasticity to concurrently provide strength and adjustability for the outer layer 102.

The emulsion pad 104 may be constructed of any sterile, non-stick material that can support the treatment material 106. As a non-limiting example, the emulsion pad 104 may be a fabric or polymer that may, or may not, be absorbent. By constructing the emulsion pad 104 of a woven fabric, the treatment material 106 can be evenly suspended and applied to a burned region while allowing easy removal due to the treatment material 106 soaking into the emulsion pad 104. It is contemplated that multiple different emulsion pad 104 materials can be utilized in the burn bandage system 100 to supply similar, or dissimilar treatment materials 106 with different application specifications, such as pad 104 density, absorbency, and thickness, which can alter the amount of treatment material 106 available and the amount of pressure to be applied to a burned region. It is noted that the outer layer 102 can be sealed by a backing layer 112 to enclose the emulsion pad 104 and treatment material 106 as a bandage assembly for later use

FIGS. 2A and 2B respectively display top and bottom line representations of an example burn bandage assembly 120 arranged in accordance with some embodiments. The bottom view of FIG. 2A shows how the burn bandage assembly 120 employs multiple separate adhesive strips 122 that can provide a waterproof seal for the outer layer 102 against a patient's skin. The emulsion pad 104 is illustrated as a cross-hatched region that may correspond with a woven fabric that continuously spans across the alignment window 124.

The top view of FIG. 2B shows how the alignment window 124 is partially, or completely, transparent to allow the emulsion pad 104 and underlying burned region of a patient's skin to be visible. The alignment window 124 can be customized for shape and material by removing portions of the outer layer 102 to optimize the efficiency and accuracy of placing the burn bandage assembly 120 to surround and seal a burn injury. That is, a user can selectively remove segmented regions of the outer layer 102 to manipulate the shape and size of the alignment window 124 to accommodate a diverse variety of burns. As such, it is noted that the outer layer 102 may be any size and material to ensure complete coverage of a burned region as well as the application of a predetermined amount of pressure to maintain contact of the emulsion pad 104 with the burned region after the outer layer 102 is affixed to the patient.

FIG. 3 is a top view line representation of another example burn bandage assembly 130 that is configured in accordance with various embodiments. While not required or limiting, the burn bandage assembly 130 has continuously transparent outer layer 132 that is shaped like a cross or “+” sign and is adhered to a patient's skin 134 via several adhesive strips 136. The ability to attach the outer layer 132, underlying emulsion pad 104, and treatment material 106 with a variety of different means allows a burned region to be sealed with a water-tight barrier in multiple manners.

It is noted that a one or more adhesive strips/layers may be present between the outer layer 132 and the patient's skin 134. The adhesive strips 136 shown in FIG. 3 can be used as secondary or primary adhesion means and can be arranged in an unlimited variety of configurations. As shown, multiple strips 136 can span a portion of the outer layer 132, as provided in region 138. One or more circular, or semi-circular strips 136 may be extend from the outer layer 132 to the patient's skin 134, as provided in region 140. A single adhesive strip 136 may also be utilized either on an edge of the outer layer 132, as provided in region 142, or across a median portion of the outer layer 132, as provided in region 144.

It is contemplated that portions of the outer layer 132 can be designated for removal by a user to alter the shape and size of the transparent portion of the layer 132. That is, some, or all, of a semitransparent portion of the outer layer 132 may be initially covered with opaque material that is segmented into two or more smaller portions, such as via perforations 146, which can be individually, or collectively, removed by a user to provide a semitransparent region as designated by the user. The ability to selectively remove portions of the outer layer 132 to reveal semitransparent regions allows a user to adjust the bandage system 130 to adapt to the size and shape of a patient's wound. With a variety of different adhesion positions and configurations, the burn bandage assembly 130 can be quickly and accurately secured about a burned region without concern for delamination of the outer layer 132 from the patient's skin 134.

It is contemplated that the application of the adhesive strip(s) 136 can be cumbersome and time-consuming. Accordingly, the example burn bandage assembly 150 of FIG. 4 illustrates how a compressive feature 152 can surround an outer layer 154 and apply continuous pressure onto the layer 154 and the patient's skin 134. In some embodiments, the compressive feature 152 is an elastic sleeve that fits around an appendage of the patient and applies a uniform pressure across the octagonal shape of the outer layer 154. The shape and size of the outer layer 154 may be selected to optimize the compressive force onto the emulsion pad 104 and treatment material 106.

In other embodiments, the compressive feature 152 is a bracelet or bladder that spans the outer layer 154 and applies uniform compressive force. For a bladder embodiment, such as a blood pressure cuff, the compressive force may be adjusted manually or automatically to ensure continuous pressure on a burned region, which may mitigate trauma during transport. While a transparent window 156 is shown in outer layer 154, the use of the compressive feature 152 may correspond with an opaque outer layer 154.

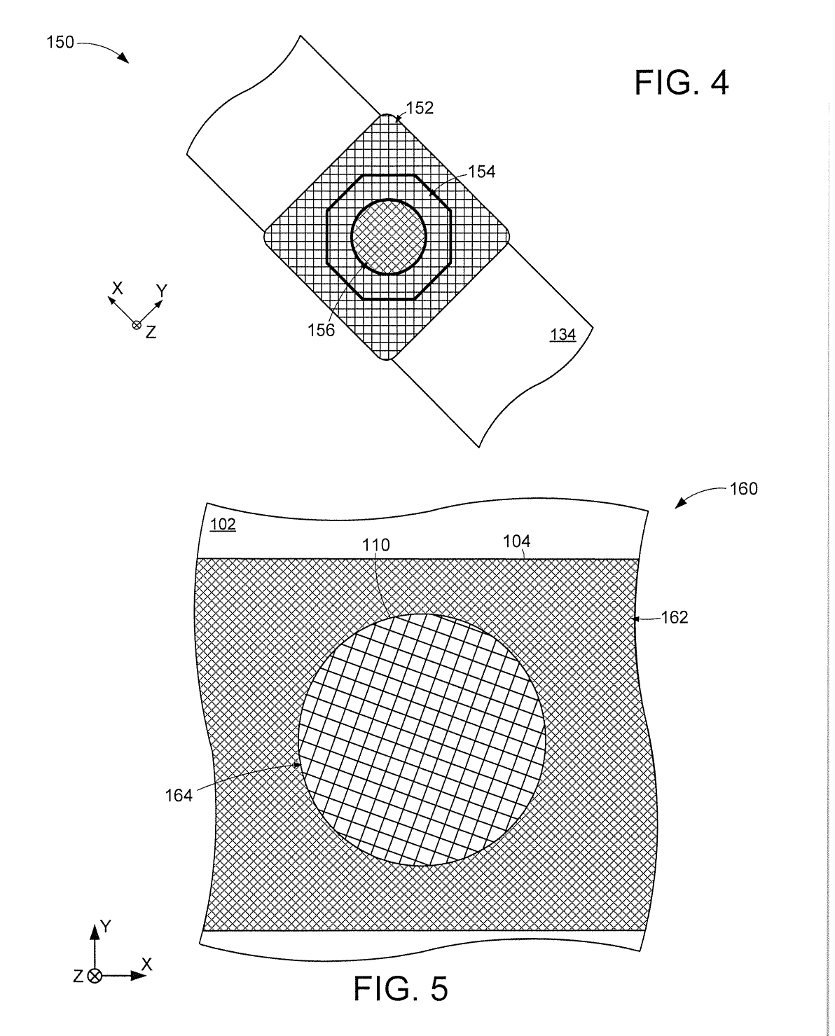

FIG. 5 displays a bottom view line representation of a portion of an example burn bandage assembly 160 constructed and operated in accordance with various embodiments. The bandage assembly 160 has an emulsion pad 104 configured with a multitude of different characteristics to allow the outer layer window 110 to be more accurately utilized. The emulsion pad 104 employs a high density material 162 away from the outer layer window 110 and a lower density material 164 underneath the window 110. It is noted that the different densities and materials of the emulsion pad 104 are not required and a common material and/or density can be arranged differently, such as with a larger weave pattern.

The decreased density beneath the outer layer window 110 allows a user to more completely see where the burn bandage assembly 160 is being positioned compared to an emulsion pad 104 having a single density and opacity. The multiple different regions of the emulsion pad 104 may also customize the application of a treatment material 106 onto a burn region. That is, a heavier, thicker, or denser emulsion pad region can transmit more pressure to desired portions of a burned region, such as the periphery of the region while the lighter, thinner, or less dense portions of the emulsion pad 104 can more gently apply the treatment material to the burned region.

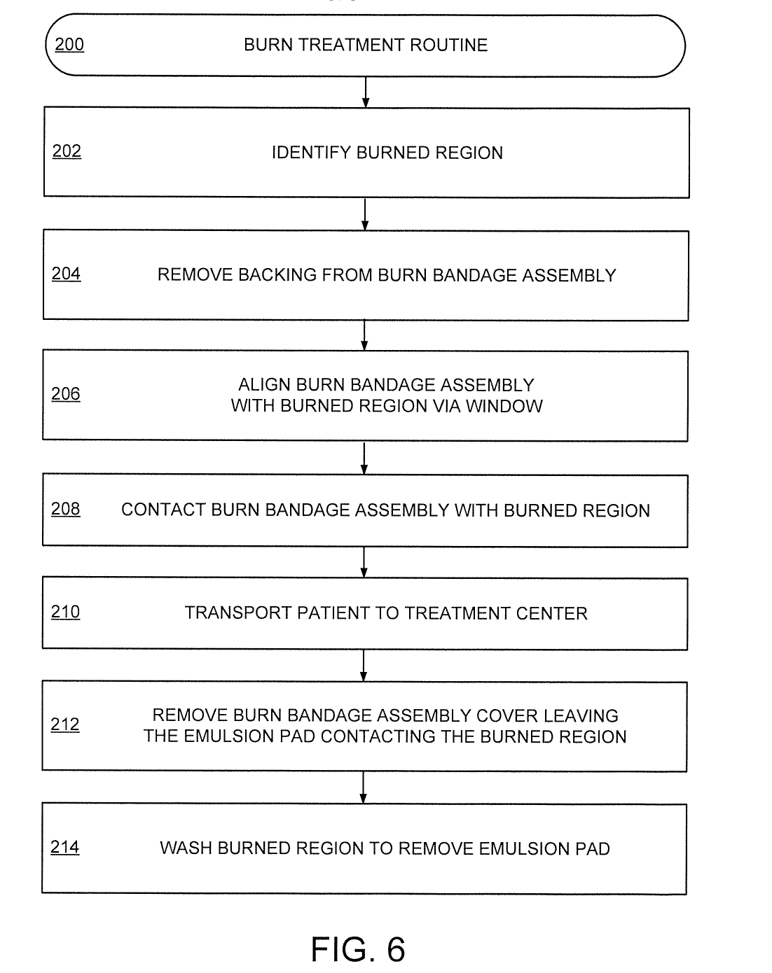

FIG. 6 provides an example burn treatment routine 200 that can be carried out with one or burn bandage assemblies as part of a burn treatment system. Initially, a burned region of a patient is identified in step 202 prior to a pre-packaged burn bandage assembly being located and step 204 proceeding to remove a protective backing. The pre-packaging of the burn bandage assembly together into a sterile arrangement allows for simple removal of a backing, such as a paper, fabric, or seal, and efficient preparation of the burn bandage assembly that minimizes a burned region's exposure. It is contemplated that the removal of the backing in step 204 activates one or more adhesive layers and/or a treatment material.

With the burn bandage assembly prepared, step 206 aligns the burn bandage assembly with the burned region of a patient's skin via one or more alignment windows in the outer layer of the burn bandage assembly, such as window 110 of FIG. 1. Alignment of the outer layer may consist of positioning the window(s) directly over the burned region so that the outer layer continuously spans and surrounds the burned region. Once aligned, step 208 then contacts the bandage assembly around the burned region, which may involve the adhesion of one or more adhesive strips between the outer layer of the bandage assembly and the patient's skin as well as the secondary use of at least one adhesive strip atop of the outer layer, as illustrated in FIG. 3.

It is noted that the contact of the burn bandage assembly in step 208 seals the assembly to the patient's skin and creates a water-tight environment around the burned region where a predetermined amount of uniform or varied continuous pressure maintains contact of the treatment material and the burned region. While the treatment material is moisturizing the burned region and the outer layer is protecting the burned region from contamination and trauma, step 210 transports the patient to a treatment center where the outer layer of the burn bandage assembly is removed in step 212. The removal of the outer layer may be conducted in any number of ways, such as washing, peeling, or dissolving the adhesive ingredients of the adhesive strips, without moving or removing any portion of the emulsion pad that continues to supply treatment material to the burned region.

The maintenance of the emulsion pad and treatment material after the outer layer has been removed allows medical personnel to assess the burned region, diagnose the severity of the injury, and strategize a treatment protocol without having to worry about the burned region becoming dry. Such added treatment time contrasts a conventional burn bandage where time is of the essence and proper diagnosis and treatment is in jeopardy due to the burned region worsening. Hence, the burn bandage assembly's application of treatment material in the field can slow and/or stop the spread and severity of a burn injury.

When a treatment protocol is determined, step 214 proceeds to wash and remove the emulsion pad from the burned region. Step 214 may be conducted with more than one solvent that does not harm the underlying burned region. In various embodiments, the removal of the emulsion pad in step 214 does not remove all the treatment material, which may be a petroleum based product that does not dissolve in the presence of water.

Through the various embodiments of the burn bandage system, a burn bandage assembly can be efficiently prepared, applied, and removed to mitigate damage from an inflicted burn or skin injury. The ability to customize the materials, configurations, and pressure applied to a burned region by a burn bandage assembly allows the system to be utilized in a diverse variety of situations, environments, and patients. The increased speed of preparation and application for the burn bandage assembly can save precious moments and provide treatment material to an injured area that can mitigate the severity of the injury. It is noted, however, that the assorted embodiments are not limited or required and can be altered in form and/or function without deterring from the spirit of the present disclosure.

What is claimed is:

1. An apparatus comprising:
an outer layer having a window portion, the outer layer comprising a first material and the window portion comprising a semitransparent second material;
an inner layer suspended across the window portion, the inner layer coated with at least one medication material; and
an adhesive layer disposed between and contacting the outer layer and a skin of a patient to apply continuous pressure on the inner layer.

2. The apparatus of claim 1, wherein the translucent second material is sterile.

3. The apparatus of claim 1, wherein the adhesive layer comprises an adhesive material configured to temporarily outer layer to the patient.

4. The apparatus of claim 1, wherein the window portion continuously surrounds a wound of the patient with a watertight seal.

5. The apparatus of claim 4, wherein the adhesive layer surrounds the wound without contacting the wound.

6. The apparatus of claim 1, wherein the first and second materials are different.

7. The apparatus of claim 1, wherein the at least one medication material is silver chloride.

8. The apparatus of claim 1, wherein the semitransparent second material is tinted.

9. An apparatus comprising
a backing layer affixed to an outer layer via at least one adhesive layer, the outer layer having a window portion and comprising a first material, the window portion comprising a semitransparent second material; and
an inner layer suspended between the backing layer and the window portion, the inner layer coated with at least one medication material, the at least one adhesive layer configured to temporarily affix the outer layer to a skin of a patient to apply pressure on the inner layer.

10. The apparatus of claim 9, wherein the inner layer has multiple different densities.

11. The apparatus of claim 10, wherein a lower density portion of the inner layer is proximal the window portion and a higher density portion of the inner layer is distal the window portion.

12. The apparatus of claim 9, wherein the outer layer contacts a compression member that applies continuous pressure on the outer layer, window portion, and inner layer concurrently.

13. The apparatus of claim 12, wherein the compression member comprises an inflatable bladder.

14. The apparatus of claim 12, wherein the compression member comprises a sleeve.

15. The apparatus of claim 9, wherein the inner layer is coated with two different materials.

16. A method comprising:
providing a bandage assembly comprising an inner layer suspended between a backing layer and a window portion of an outer layer, the outer layer comprising a first material and the window portion comprising a semitransparent second material, the inner layer coated with at least one medication material;
removing the backing layer to reveal an adhesive layer contacting a periphery of the outer layer;
aligning the window portion of the outer layer with a wound of a patient; and
affixing the adhesive layer to a skin of the patient to create a waterproof seal around the wound with the inner layer in contact with the wound, the adhesive and outer layers configured to apply continuous pressure on the inner layer towards the wound.

17. The method of claim 16, wherein the outer and adhesive layers are removed subsequent to the affixing step without removing the inner layer.

18. The method of claim 16, wherein the inner layer is configured to be removed by washing.

19. The method of claim 16, wherein the at least one medication material continually provides moisture to the wound.

20. The method of claim 16, wherein an inflatable bladder is applied to the outer layer and inflated to apply pressure onto the inner layer in addition to the pressure applied by the outer and adhesive layers.
